(19) United States
(12) Patent Application Publication
(10) Pub. No.: US 2015/0216908 A1
(43) Pub. Date: Aug. 6, 2015

(54) QUADRI-POSITIVE STROMAL CELL (QPSC) POPULATION FOR SUPERIOR CELL PROTECTION AND IMMUNOMODULATION

(71) Applicant: STEMINENT BIOTHERAPEUTICS INC., Taipei City (TW)

(72) Inventors: Oscar Kuang-Sheng Lee, Taipei City (TW); Jennifer Hui-Chun Ho, Taipei City (TW); Wei-Kee Ong, Taipei City (TW); Yu-Hsuan Chu, Taipei City (TW)

(73) Assignee: STEMINENT BIOTHERAPEUTICS INC., Taipei City (TW)

(21) Appl. No.: 14/615,737

(22) Filed: Feb. 6, 2015

Related U.S. Application Data
(60) Provisional application No. 61/936,407, filed on Feb. 6, 2014.

(51) Int. Cl. A61K 35/35; C12N 5/077

(52) U.S. Cl. CPC A61K 35/35; C12N 5/0652

(57) ABSTRACT

The invention unexpectedly found that an isolated and modified QPSC population has multi-potentiality, including: osteoblasts (bone cells), chondrocytes (cartilage cells), and adipocytes (fat cells). The QPSC population of the invention features a desirable immunomodulation ability, including inducing, enhancing, or suppressing an immune response and thus has potential value in the prevention and/or treatment of various immune diseases/disorders/conditions. The invention has effective homing ability and regulation ability in complement-dependent cytotoxicity, including the ability to block the activation of host complements and direct migration to the target area and the ability to enhance cell viability, and thus offers better cell protection and therapeutic efficacy in vivo in the prevention and/or treatment of various acute tissue injury, ischemic or degenerative diseases/disorders/conditions.

### CROSS-REFERENCE TO RELATED APPLICATIONS

This application claims priority to U.S. Provisional Ser. No. 61/936,407, filed Feb. 6, 2014, which is incorporated herein by reference in its entirety.

### FIELD OF THE INVENTION

The invention relates to an isolated and modified population of human multipotent stromal cells (hereafter referred to as Quadri-Positive Stromal Cell (QPSC) population), methods for preparing and/or applying the same and compositions comprising the same. In particular, the invention provides CD273 co-expressing with CD55, CD46 and/or CXCR4 markers on QPSC population.

### BACKGROUND OF THE INVENTION

Stem cell transplantation is known to possess the potential for regenerating damaged or injured tissues and organs. Consequently, intensive studies have been carried out in order to identify and isolate new multipotent stem cells from both human and animal origin, as well as to investigate the effectiveness of stem cells in clinical applications, especially regenerative medicine. Multipotent stromal cells, or MSCs, possess at least tri-lineage differentiation capacity, i.e., osteogenic (osteoblasts), chondrogenic (chondrocytes), and adipogenic (adipocytes) differentiation. There is documented evidence of therapeutic efficacy of MSCs in treating musculoskeletal injuries, improving cardiac function in the cardiovascular disease and ameliorating the severity of GVHD. Under adequate induction, they have the potential to differentiate into not only mesenchymal, but also ectodermal and endodermal cell types without teratoma formation. MSCs lack immunogenecity but with immunomodulatory ability, so host immune rejection of transplanted MSCs is routinely circumvented through autologous or allogeneic transplantation. MSCs can be isolated from several adult tissues, including bone marrow (BM), adipose tissues (AD), and cord blood, and can be expanded ex vivo. For example, mesenchymal stem/progenitor cells have been successfully isolated from umbilical cord tissue, namely from Wharton's jelly, the matrix of umbilical cord (U.S. Pat. No. 5,919,702 and US Patent Application 2004/0136967). These cells have been shown to have the capacity to differentiate, for example, into a neuronal phenotype and into cartilage tissue, respectively. Furthermore, mesenchymal stem/progenitor cells have also been isolated from the endothelium and the subendothelial layer of the umbilical cord vein, one of the three vessels (two arteries, one vein) found within the umbilical cord. The International Society for Cellular Therapy (ISCT) has proposed a set of standards to define MSCs. A cell can be classified as an MSC if it shows plastic adherent properties under normal culture conditions and has a fibroblast-like morphology. Furthermore, MSCs can undergo osteogenic, adipogenic and chondrogenic differentiation ex-vivo. The cultured MSCs also express on their surface CD73, CD90 and CD105, while lacking the expression of CD11b, CD14, CD19, CD34, CD45, CD79a and HLA-DR surface markers (Dominici, M et al., “Minimal criteria for defining multipotent mesenchymal stromal cells. The International Society for Cellular Therapy position statement.” 2006, Cytotherapy Vol. 8, No. 4, pp. 315-317).

US 20100184033 A1 relates to methods to differentiate pluripotent primordial stem cells, such as human embryonic stem (“hES”) cells, human embryonic germ (“hEG”) cells, human embryo-derived (“hED”) cells and human embryonal carcinoma (“hEC”) cells, to obtain subpopulations of cells from heterogeneous mixtures of cells, wherein some stem cells express CD273 antigen. However, the stem cells are not MSCs.

US 20120171171 A1 provides embryonic chondrocyte progenitor cells or cell lines which are capable of generating cartilage without expressing COL10A1 or the IHH gene and are negative for the expression of any one, two, three, four or all of the following genes: CD74, CD90, CD166, ITGA2, and KCNK2. Although the application mentions that cells thereof include 40.48% of CD273-positive cells, they are not MSCs.

However, there is a need to find new multipotent stem cells with superior cell protection and immunomodulation ability (especially in vivo) to increase the therapeutic efficacy in tissue repair and regeneration.

### SUMMARY OF THE INVENTION

The invention provides an isolated and modified QPSC population, which has at least 70% cell homogeneity and expresses at least a cell marker of CD273. In one embodiment, in addition to CD273, the QPSC population further expresses one or more of CD55, CD46 and CXCR4. Particularly, the QPSC population of the invention can strongly express CD273, CD73, CD105 and CD90 and CD55, CD46 and CXCR4 while not expressing one or more of CD11b, CD19, CD34, Cd45 and HLA-DR.

The QPSC population of the invention has at least 70% cell homogeneity and appears to express at least a cell marker of CD273 after cultivation to at least 4th, 5th, 6th, 7th, 8th or 9th passage and maintains the cell marker expression after at least the 4th, 5th, 6th, 7th, 8th or 9th passage, preferably expression and maintenance of the cell marker from the 4th passage to 15th passage or from the 5th passage to 15th passage, from the 6th passage to 15th passage or from the 6th passage to 14th passage, from the 6th passage to 13th passage, from the 6th passage to 12th passage, from the 8th passage to 15th passage or from the 8th passage to 12th passage. Preferably, the QPSC population of the invention maintains the expression of at least CD273 after the 6th passage to 12th, 13 th, 14 th, 15th, 16th, 17th or 18th passage.

In some embodiments, the QPSC population of the invention appears to express CD273 and optionally one or more of CD46, CD55 and CXCR4 after cultivation to at least the 4th, 5th, 6th, 7th, 8th or 9th passage and maintains the CD273 expression and one or more of CD46, CD55 and CXCR4 after at least the 4th, 5th, 6th, 7th, 8th or 9th passage; preferably expression and maintenance of the cell marker(s) from the 4th passage to 15th passage or from the 5th passage to 15th passage, from the 6th passage to 15th passage or from the 6th passage to 14th passage, from the 6th passage to 13th passage, from the 6th passage to 12th passage, from the 8th passage to 15th passage or from the 8th passage to 12th passage. More preferably, the QPSC population of the invention appears to express CD273, CD46, CD55 and CXCR4 after cultivation to at least the 5th, 6th, 7th, 8th or 9th passage and maintains the expression of CD273 CD46, CD55 and CXCR4 after at least the 5th, 6th, 7th, 8th or 9th passage, preferably from the 4th passage to at least the 15th passage, from the 4th passage to 15th passage or from the 5th passage to 15th passage, from the 6th passage to 15th passage or from the 6th passage to 14th passage, from the 6th passage to 13th passage, from the 6th passage to 12th passage, from the 8th passage to 15th passage or from the 8th passage to 12th passage. Preferably, the QPSC population of the invention maintains the expression of CD273 and one or more of CD46, CD55 and CXCR4 after the 6th passage to 12th, 13 th, 14th, 15th, 16th, 17th or 18th passage.

In another aspect, the invention provides a composition comprising the QPSC population of the invention.

In one aspect, the invention provides a method for producing an isolated and modified QPSC population of the invention having at least 70% cell homogeneity, comprising isolating human multipotent stromal cells from a subject; culturing the resulting cells in a stem cell culture medium; detaching the cells after they reach 70-80% confluence; seeding the cells at a density in the range of 3,000-10,000 cells/cm², preferably 5,000 to 10,000 cells/cm², 6,000 to 10,000 cells/cm² or 7,000 to 10,000 cells/cm²; culturing the cells in a and a medium supplement (preferably ITS (a mixture of insulin, transferrin, selenous acid, BSA, and linoleic acid; preferably 0.1-1%) containing at least one growth factor (preferably, EGF (preferably 0.1-1.0 ng/ml), FGF-2 (preferably 1-10 ng/ml); and after cultivation to at least the 6th passage, at least 70% cells appear to express CD273 and optionally one or more of CD46, CD55 and CXCR4 and maintaining the expression of CD273 and optionally one or more of CD46, CD55 and CXCR4 from at least the 6th passage to at least the 12th passage, whereby an isolated and modified QPSC population can be obtained. Preferably, the medium further comprises a medium supplement (preferably ITS (a mixture of insulin, transferrin, selenous acid, BSA, and linoleic acid. Preferably, the medium supplement is in an amount of 0.1-1%.

In another aspect, the invention provides a method for modulation of T cells and enhancement of cell viability, comprising administering an effective amount of the isolated and modified QPSC population of the invention to a subject. In a further aspect, the invention provides a method for prevention and/or treatment of an immune/ischemic disease, disorder or condition, comprising administering an effective amount of the isolated and modified QPSC population of the invention to a subject.

### BRIEF DESCRIPTION OF THE DRAWING

FIG. 1(A) shows morphology of the cells of the QPSC population.

FIG. 1(B) shows morphology of the cells of the QPSC population in 80% confluence.

FIG. 2(A) shows surface phenotype of the cells of the QPSC P10 populations (QP marker first).

FIG. 2(B) shows surface phenotype of the cells of the QPSC P12 populations.

FIG. 3(A) shows tri-lineage differentiation of the cells of the QPSC population in osteogenesis (alkaline phosphate).

FIG. 3(B) shows tri-lineage differentiation of the cells of the QPSC population in osteogenesis (calcium).

FIG. 3(C) shows tri-lineage differentiation of the cells of the QPSC population in chondrogenesis.

FIG. 3(D) shows tri-lineage differentiation of the cells of the QPSC population in adipogenesis.

FIG. 4 shows CD3+ T-cell proliferation ability of QPSCs. To examine the QPSC immunomodulatory effect on T lymphocyte proliferation, QPSCs were co-cultured at various ratios with freshly isolated T lymphocytes stimulated by plate-bound anti-CD3 antibody plus anti-CD28 antibody. T cell proliferation was then measured using BrdU incorporation method. BrdU (5-bromo-2′-deoxyuridine) is a synthetic thymidine analogue that can be incorporated into newly synthesized DNA in proliferating cells and detected by immunoassay. The results show that T cell proliferation was significantly inhibited by the QPSCs but not in a cell-number-dependent manner.

FIG. 5(A) shows CD273 on QPSCs for T-cell regulation (the cells of QPSC population, passage 10).

FIG. 5(B) (the cells of QPSC population, passage 12) shows that the ability to inhibit T cell proliferation of CD273⁻QPSC population was significantly reduced. In addition, CD273⁻QPSCs were purified by negative selection using anti-human CD273 antibody conjugated magnetic particles.

FIG. 5(C) shows that CD273⁻QPSC population failed to suppress T cell proliferation. These results indicate that CD273 expression is important for the immunomodulatory function of the cells of QPSC population.

FIG. 6(A) shows cell marker profile of the cells of QPSC population p10 after 4 weeks cryopreservation and then thawing.

FIG. 6(B) shows cell marker profile of the cells of QPSC population p12 after 4 weeks cryopreservation and then thawing.

FIG. 7(A) shows CXCR4 on the cells of QPSC population for homing wherein CXCR4^{High} MSCs (QPSCs, n=2) and CXCR4^{Low} MSCs (n=3) migrate toward SDF-1.

FIG. 7(B) shows Bar Chart of the homing.

FIG. 8(A) shows CD46 and CD55 on QPSC population for the ability of resisting complement-dependent cytotoxicity.

FIG. 8(B) shows cell viability of MTS assay.

### DETAILED DESCRIPTION OF THE INVENTION

The invention unexpectedly found that isolated and modified QPSC population having multi-potentiality, including: osteoblasts (bone cells), chondrocytes (cartilage cells), and adipocytes (fat cells). The QPSC population of the invention has immunomodulative ability to modulate T cells and/or complement system and has the ability to enhance cell viability, suggesting that they induce, enhance, or suppress an immune response and enhance homing efficiency as well, and thus have potential value in the prevention and/or treatment of various immune and ischemic diseases, disorders or conditions.

### DEFINITIONS

As used herein, “stem cells” are cells that possess self-renewal ability and multiple differentiation ability when exposed to specific environmental conditions. Self-renewal means that during cell division, at least one of the two daughter cells will be a stem cell.

As used herein, “multi-potent” means a cell that has the potential of differentiating into at least three cell types.

As used herein, “differentiation” means the formation of cells expressing functional markers known to be associated with cells that are more specialized and closer to becoming terminally differentiated cells incapable of further division or differentiation.

As used herein, “autologous transplant” means that the transplanted material is derived from and transplanted to the same individual.

As used herein, “proliferation” or “expansion” means an increase in cell number.

As used herein, “cell surface marker” means a protein expressed on the surface of a cell which is detectable via specific antibodies.

As used herein, “positive for expression” means that the marker of interest, whether intracellular or extracellular, is detectable in or on a cell using any method, including but not limited to flow cytometry. The terms “positive for expression,” “positively expressing,” “expressing,” and “+” used in superscript are used interchangeably herein.

As used herein, “negative for expression” means that the marker of interest, whether intracellular or extracellular, is not detectable in or on a cell using any method, including but not limited to flow cytometry. The terms “negative for expression,” “negative expressing,” “not expressing,” and “−” used in superscript are used interchangeably herein.

As used herein, “isolated,” used in reference to a single cell or cell population, means that the cell or cell population is substantially free of other cell types or cellular material with which it naturally occurs in its or their origin.

As used herein, “modified” means that the naturally isolated cells are modified to have markedly different characteristics such as different structure, different biological or pharmacological function and/or other properties.

As used herein, the term “cell population” means a population of cells that is substantially homogenous. A population of cells that are at least 70%, 75%, 80%, 85%, 90%, 91%, 92%, 93%, 94%, 95%, 96%, 97%, 98% or 99% is homogenous in cell marker expression.

As used herein, the term “strongly express” or “strong expression” refers to expression intensity of a cell marker over 70%; the term “moderately express” or “moderate expression” refers to expression intensity of a cell marker over 40%.

As used herein, “immunomodulation” and “immunomodulatory” mean causing, or having the capacity to cause, a detectable change in an immune response.

As used herein, “immunosuppression” and “immunosuppressive” mean causing, or having the capacity to cause, a detectable reduction in an immune response, and the ability to cause a detectable suppression of an immune response.

As used herein, “complement system” is a part of the immune system that helps or complements the ability of antibodies and phagocytic cells to clear pathogens from an organism.

As used herein, “complement C3” is a blood test that measures the activity of a certain protein that is part of the complement system. The complement system is a group of proteins that move freely through the bloodstream. The proteins work with the immune system and play a role in the development of inflammation.

As use herein, “homing” is the phenomenon whereby cells migrate to the organ of their origin or specialized niches, i.e., areas of injury.

### Isolated and Modified QPSC Cell Population and Composition Comprising the Same

In one aspect, the invention provides an isolated and modified QPSC population, which has at least 70% cell homogeneity and expresses at least a cell marker of CD273, preferably further expresses one or more of CD55, CD46 and CXCR4. In one embodiment, in addition to CD273, the QPSC population strongly expresses CD73, CD105 and CD90, while not expressing one or more of CD11b, CD19, CD34, Cd45 and HLA-DR.

Antibodies can be used to recognize surface molecules differentially expressed on target cells. The cell surface marker means a protein expressed on the surface of a cell, which is detectable via specific antibodies. Cell markers as well as surface markers that are useful in the invention include, but are not limited to, the CD (clusters of differentiation) antigens CD73, CD105, CD90, CD273, CD46, CD55, CXCR4, CD11b, CD19, CD34, CD45 and HLA-DR. The above markers are illustrated as follows.

CD73 (NTSE, ecto-5′-nucleotidase, ESNT, NTE) is a glycosyl phosphatidylinositol (GPI)-anchored purine salvage enzyme expressed on the surface of human T and B lymphocytes. CD73 plays a role in activating T cells.

CD90 is a GPI-cell anchored molecule found on prothymocyte cells.

CD105 is a disulfide-linked homodimer found on endothelial cells but absent from most T and B cells.

CD273 is a second ligand (co-ligand) for PD1 (co-receptor) that is important for regulation of T cell activation and is known to be expressed in DC and macrophage.

CD55 is a complement decay-accelerating factor that regulates the complement system on the cell surface and prevents the assembly of the C3bBb complex (the C3-convertase of the alternative pathway) or accelerates the disassembly of preformed convertase, thus blocking the formation of the membrane attack complex; CD46 is an inhibitory complement receptor.

CXCR4 is broadly expressed in cells of both the immune and the central nervous systems, mediates migration of resting leukocytes and haematopoietic progenitors in response to its ligand, SDF-1(CXCL12), and one of the main functions of this SDF-1/CXCR4 axis is the regulation of progenitor cell trafficking during embryonic development, cell chemotaxis, and postnatal homing into injury sites.

CD11b is the integrin alpha M chain; CD11c is the integrin alpha X chain and is important for the adherence of neutrophils and monocytes to stimulated endothelium, and also plays a role in phagocytosis of complement coated particles and as a marker to distinguish between recently activated effector CD8(+) T cells and memory cells.

CD19 is found on the surface of B-cells, a type of white blood cell, and is expressed on follicular dendritic cells and B cells.

CD34 is a cluster of differentiation molecule present on certain cells within the human body and a cell surface glycoprotein and functions as a cell-cell adhesion factor; it may also mediate the attachment of stem cells to bone marrow extracellular matrix or directly to stromal cells.

CD45 is a leukocyte common antigen found on all cells of hematopoietic origin.

HLA-DR is the MHC Class II molecule.

In one embodiment, the QPSC population of the invention has at least 70% cell homogeneity and can strongly express at least CD273, CD73, CD105 and CD90. In addition to CD273, CD73, CD105 and CD90, the QPSC population of the invention can strongly express one or more of CD55, CD46 and CXCR4. In another embodiment, the QPSC population of the invention can strongly express CD90, CD105, CD73, CD273, CD46, CD55 and CXCR4.

In some embodiments, the lack of expression of a cell surface marker defines the QPSC population of the invention. According to the invention, the QPSC population has at least 70% cell homogeneity and is negative for one or more of CD11b, CD19, CD34, CD45 and HLA-DR. Based on the above-mentioned unique cell surface marker signatures, individual stem cell populations having unique functional characteristics have been identified.

In some embodiments, at least about 70%, about 75%, about 80%, about 85%, about 90%, about 91%, about 92%, about 93%, about 94%, about 95%, about 96%, about 97%, about 98%, or about 99% of the QPSCs within a cell population of the invention express the cell markers of interest; in other embodiments at least about 85%, about 86%, about 87%, about 88%, about 89%, about 90%, about 91%, about 92%, about 93%, about 94%, about 95%, about 96%, about 97%, about 98%, or about 99% of the QPSC population within the stem cell population express the cell markers of interest; in yet other embodiments at least about 95%, about 96%, about 97%, about 98%, about 99%, or even about 100% of the QPSC population within the stem cell population express the cell markers of interest. “Substantially free” means less than about 30%, 25%, 20%, 15%, 10%, 9%, 8%, 7%, 6%, 5%, 4%, 3%, 2%, 1%, or even 0% of the cells in the population expressing the marker of interest.

In one embodiment, the QPSC population of the invention has at least 70% cell homogeneity and appears to express at least a cell marker of CD273

after cultivation to at least 4^{th}, 5^{th}, 6^{th}, 7^{th}, 8^{th} or 9th passage and maintains the cell marker expression after at least the 4^{th}, 5^{th}, 6^{th}, 7^{th}, 8^{th} or 9th passage, preferably expression and maintenance of at least CD273 from the 4^{th} passage to 15th passage or from the 5^{th} passage to 15th passage, from the 6^{th} passage to 15th passage or from the 6^{th} passage to 14th passage, from the 6^{th} passage to 13th passage, from the 6^{th} passage to 12th passage, from the 8^{th} passage to 15th passage or from the 8^{th} passage to 12th passage. Preferably, the QPSC population of the invention maintains the expression of at least CD273 after the 6th passage to 12^{th}, 13 th, 14 th, 15^{th}, 16th, 17^{th} or 18th passage.

In some embodiments, the QPSC population of the invention appears to express CD273 and optionally one or more of CD46, CD55 and CXCR4 after cultivation to at least the 4th, 5th, 6th, 7th, 8th or 9th passage and maintains the CD273 expression and one or more of CD46, CD55 and CXCR4 after at least the 4^{th}, 5th, 6th, 7th, 8th or 9th passage; preferably expression and maintenance of the cell marker(s) from the 4^{th} passage to 15th passage or from the 5^{th} passage to 15th passage, from the 6^{th} passage to 15th passage or from the 6^{th} passage to 14th passage, from the 6^{th} passage to 13th passage, from the 6^{th} passage to 12th passage, from the 8^{th} passage to 15th passage or from the 8^{th} passage to 12th passage. More preferably, the QPSC population of the invention appears to express CD273, CD46, CD55 and CXCR4 after cultivation to at least the 5th, 6th, 7th, 8th or 9th passage and maintains the expression of CD273 CD46, CD55 and CXCR4 after at least the 5th, 6th, 7th, 8th or 9th passage, preferably from the 4th passage to at least the 15th passage, from the 4^{th} passage to 15th passage or from the 5^{th} passage to 15th passage, from the 6^{th} passage to 15th passage or from the 6^{th} passage to 14th passage, from the 6^{th} passage to 13th passage, from the 6^{th} passage to 12th passage, from the 8^{th} passage to 15th passage or from the 8^{th} passage to 12th passage. Preferably, the QPSC population of the invention maintains the expression of CD273 and one or more of CD46, CD55 and CXCR4 after the 6th passage to 12^{th}, 13 th, 14 th, 15^{th}, 16th, 17^{th} or 18th passage.

In one embodiment, the QPSC population of the invention remains substantially identical in expression profile after cryopreservation and thawing.

Selective methods known in the art and described herein can be used to characterize QPSC population. Commonly, sources of QPSC population are reacted with monoclonal/polyclonal antibodies. Subpopulations of cells expressing cell surface antigens can be labeled by fluorescence materials such quantum dots, or labeled by secondary antibodies or be either positively or negatively selected by immunomagnetic bead elucidation, complement mediated lysis, agglutination methods, commercial antibody array kits or fluorescence activated cell sorting (FACS). The functional attributes of the resulting subpopulations with a defined cell surface phenotype are then determined using a colony-forming assay.

In one embodiment, the QPSC population of the invention has at least 3-fold superior anti-oxidation ability relative to human corneal epithelial cell.

In one embodiment, the QPSC population of the invention expresses genes of growth factors (preferably EGF, VEGF), immunomodulation (preferably IDO, IL10, TGF-β1), and neurotrophic factors (preferably GDNF, CNTF, NGF, NTF3, NTF4).

In one embodiment, the QPSC population of the invention synthesizes and secretes cytokines, including angiopoietin-2, BDNF, BTC, FGF-4, FGF-9, IGF-II, IL3, IL12-p40, GRO, MCP-1, MIF, MIP-1α, Fas/TNFRSF6, IGF-BP-3, TRAIL-R4, TIMP-1, TIMP-2, and uPAR.

In another aspect, the invention provides a composition comprising the QPSC population of the invention. According to the invention, in addition to the QPSC population, the above composition may contain one or more inactivated carriers that are permitted pharmaceutically. Examples of the inactivated carriers include preservative, solubilizer, stabilizer, etc. The composition may be used for non-oral administration, for example intravenous, subcutaneous, intra-peritoneal administration or topical application. A dosage of the cell population may vary in accordance with kind of disease, degree of seriousness of disease, administration route, or weight, age and sex of patient.

### Process for Production of QPSC Population of the Invention

The QPSC population of the invention is cultured by maintaining it in an appropriate medium, detaching it from the medium after it reaches a proper confluence, collecting the detached cells and then seeding them in a cell culture container.

In one aspect, the invention provides a method for producing an isolated and modified QPSC population of the invention having at least 70% cell homogeneity, comprising isolating human multipotent stromal cells from a subject; culturing the resulting cells in a stem cell culture medium; detaching the cells after they reach 70-80% confluence; seeding the cells at a density in the range of 3,000-10,000 cells/cm², preferably 5,000 to 10,000 cells/cm², 6,000 to 10,000 cells/cm² or 7,000 to 10,000 cells/cm²; culturing the cells in a and a medium supplement (preferably ITS (a mixture of insulin, transferrin, selenous acid, BSA, and linoleic acid; preferably 0.1-1%) containing at least one growth factor (preferably, EGF (preferably 0.1-1.0 ng/ml), FGF-2 (preferably 1-10 ng/ml); and after cultivation to at least the 6th passage, at least 70% cells appear to express CD273 and optionally one or more of CD46, CD55 and CXCR4 and maintaining the expression of CD273 and optionally one or more of CD46, CD55 and CXCR4 from at least the 6th passage to at least the 12th passage, whereby an isolated and modified QPSC population can be obtained. Preferably, the medium further comprises a medium supplement (preferably ITS (a mixture of insulin, transferrin, selenous acid, BSA, and linoleic acid. Preferably, the medium supplement is in an amount of 0.1-1%.

In one embodiment, the QPSC population of the invention can be isolated and purified from adipose tissues using a variety of methods, including those described herein and exemplified below; they are then modified to have at least 70% cell homogeneity by enhancing and maintaining their expression on CD273 and optionally one or more of CD46, CD55 and CXCR4. For identification and characterization, the isolated and modified QPSC population is positively selected by sorting for expression of cell surface markers and negatively sorting for lack of expression of cell surface markers.

In one embodiment, the isolated and modified QPSC population of the invention can maintain expression of CD273 and optionally one or more of CD46, CD55 and CXCR4 after cryopreservation and thawing. In another aspect, the invention provides a method of maintaining cell markers of QPSC population of the invention after large-scale cell cryopreservation and thawing, comprising a step of slowly decreasing the temperature of the large-scale cells of the QPSC population at a constant cooling rate during the freezing process and then thawing, wherein the temperature difference (ΔT) between the surrounding and center of the large-scale cells of the QPSC population range from 1.6 to 2.6° C.

When freezing cells, it is necessary to decrease the temperature slowly to avoid a rapid or unstable cooling process, which may lead to cell death due to condensation of water crystals within the cell. A conventional method for cryopreservation of a living cell comprises placing the cell in a cryoprotectant; gradually cooling the living cell in cryoprotectant to a first predetermined temperature; and rapidly cooling the living cell in cryoprotectant from the first predetermined temperature to a second predetermined temperature. The step of gradually cooling the cell may include cooling at a rate of between about 1° C./min and about 3° C./min. Typically, the first predetermined temperature is about −30° C. The second predetermined temperature may be between about −80° C. and about −196° C. However, it is critical to control the temperature difference between the surrounding and center of the cells.

The invention unexpectedly found that in cryopreservatvation, controlling the temperature difference (ΔT) between the surrounding and center of cells in a large scale in a range of 1.6 to 2.6° C. when decreasing temperature can maintain cell marker expression of the cells after thawing. That is, the expression intensity of cell markers will not decrease after the frozen cells are thawed.

In one embodiment, a cryopreservation device for cryo-bag under slow cooling is used in cryopreservation of QPSC population cells of the invention. The device comprises a main body case, one or more layers of spaces for cryo-bag insertion, and two or more Teflon cryopreservation bags filled with isopropanol. The term “cryo-bag” or “cell collection bag” is defined as a cryogenic preservation bag containing viable cells. As used herein, the viable cells can be cells after in vitro culture expansion, including (but not limited to) stem cells, or the cord blood harvested from a living body. In order to avoid contamination and provide a favorable configuration for transportation and stacking, a cover lid is provided on the main body case of the cryopreservation device for controlling opening and closing by pressing-fit or snapping-fit. The main body case can be made of freeze-resistant plastic, paper, metal or other materials, and the material is not particularly limited in the present invention. In one of the embodiments, the main body case and the cover lip are made of a PP plastic material. The cryopreservation device contains a single- or multi-layer of placing space for cryo-bags. The cryo-bags can be placed in an individually removable metal case, or be set on a clapboard held by protruding objects from the inner wall of the main case. The height of the placing space should be the same as that of the cryo-bag to make sure that cryo-bag directly contacts the Teflon cryopreservative bags on the upper and lower surfaces. Preferably, the cryo-bags are intermediately stacked between the Teflon cryopreservative bags. In a better embodiment of the present invention, both upper and lower surfaces of the cryo-bag directly contact the Teflon cryopreservative bags.

### APPLICATIONS

In another aspect, the invention provides a method for modulation of T cells and/or complement system, and enhancement of cell viability, comprising administering an effective amount of the isolated and modified QPSC population of the invention to a subject. Accordingly, the QPSC population of the invention can induce, enhance, or suppress an immune response.

In another aspect, the invention provides a method for enhancement of homing efficiency of cells, comprising administering an effective amount of the isolated and modified QPSC population of the invention to a subject.

In a further aspect, the invention provides a method for prevention and/or treatment of an immune disease, disorder or condition, comprising administering an effective amount of the isolated and modified QPSC population of the invention to a subject. The isolated and modified QPSC population of the invention has immunomodulative ability, including inducing, enhancing, or suppressing an immune response, and thus has potential value in the prevention and/or treatment of various immune diseases, disorders or conditions.

In a specific embodiment, said disease, disorder or condition is an inflammatory bowel disease. In a more specific embodiment, said inflammatory bowel disease is Crohn's disease.

In another specific embodiment, said disease, disorder or condition is graft-versus-host disease. In a more specific embodiment, said graft-versus-host disease develops after an allogeneic bone marrow transplant. In another more specific embodiment, said graft-versus-host disease develops after a solid organ transplant. In another more specific embodiment, said graft-versus-host disease develops after a composite tissue allograft. In another more specific embodiment, said graft-versus-host disease is reduced in grade by at least one step by said administration.

In another specific embodiment, said disease, disorder or condition is rheumatoid arthritis (RA). In a more specific embodiment, the administration is sufficient to cause a detectable improvement in one or more symptoms of RA, or sufficient to detectably reduce the onset of one or more symptoms of RA, in at least one joint in the individual with RA. In another specific embodiment, the administration is sufficient to cause a detectable improvement in one or more symptoms of RA, or sufficient to detectably reduce the onset of one or more symptoms of RA, in at least one non-joint tissue in the individual with RA. In a more specific embodiment, said non-joint tissue is skin (dermis), lungs, autoimmune system or blood, renal tissue, cardiovascular tissue, ocular tissue, or neurological tissue. In a more specific embodiment, said symptom of RA is a condition adjunct to RA. In a more specific embodiment, said condition adjunct to RA is pyoderma gangrenosum, neutrophilic dermatosis, Sweet's syndrome, viral infection, erythema nodosum, lobular panniculitis, atrophy of digital skin, palmar erythema, diffuse thinning (rice paper skin), skin fragility, subcutaneous nodules on an exterior surface, e.g., on the elbows, fibrosis of the lungs (e.g., as a consequence of methotrexate therapy), Caplan's nodules, vascular disorders, nail fold infarcts, neuropathy, nephropathy, amyloidosis, muscular pseudohypertrophy, endocarditis, left ventricular failure, vasculitis, scleromalacia, mononeuritis multiplex, atlanto-axial subluxation.

In another specific embodiment, the disease, disorder or condition is multiple sclerosis. In a more specific embodiment, said multiple sclerosis is relapsing/remitting multiple sclerosis, secondary progressive multiple sclerosis, primary progressive multiple sclerosis, or progressive/relapsing multiple sclerosis.

In another specific embodiment, the disease, disorder or condition is lupus erythematosus. In a more specific embodiment, said symptom of lupus erythematosus is one or more of malar rash, butterfly rash, discoid lupus, alopecia, mouth, nasal, and vaginal ulcers, lesions on the skin, joint pain anemia and/or iron deficiency, lower than normal platelet and white blood cell counts, antiphospholipid antibody syndrome, presence of anticardiolipin antibody in the blood, pericarditis, myocarditis, endocarditis, lung and/or pleural inflammation, pleuritis, pleural effusion, lupus pneumonitis, chronic diffuse interstitial lung disease, pulmonary hypertension, pulmonary emboli, pulmonary hemorrhage, painless hematuria or proteinuria, lupus nephritis, renal failure, and/or development of membranous glomerulonephritis with “wire loop” abnormalities); neurological manifestations (e.g., seizures, psychosis, abnormalities in the cerebrospinal fluid); T-cell abnormalities (e.g., deficiency in CD45 phosphatase and/or increased expression of CD40 ligand); and/or nonspecific manifestations (e.g., lupus gastroenteritis, lupus pancreatitis, lupus cystitis, autoimmune inner ear disease, parasympathetic dysfunction, retinal vasculitis, systemic vasculitis, increased expression of Fc.epsilon.RI.gamma., increased and sustained calcium levels in T cells, increase of inositol triphosphate in the blood, reduction in protein kinase C phosphorylation, reduction in Ras-MAP kinase signaling, and/or a deficiency in protein kinase A I activity.

In another specific embodiment, said disease, disorder or condition is scleroderma. In a more specific embodiment, the scleroderma is diffuse scleroderma. In another specific embodiment, said disease, disorder or condition is mycosis fungoides (Alibert-Bazin syndrome). In another embodiment, said disease, disorder or condition is diabetes. In another embodiment, said disease, disorder or condition is psoriasis. In a more specific embodiment, the psoriasis is plaque psoriasis (psoriasis vulgaris).

In another specific embodiment of any of the above methods, the method comprises administration of a second therapeutic agent to the individual having the disease, disorder or condition. In a more specific embodiment, said second therapeutic agent is an anti-inflammatory agent, an immunomodulatory agent, and immunosuppressive agent, a pain medication, or an antibiotic. In a more specific embodiment, the second therapeutic agent is an immunomodulatory agent. In a more specific embodiment, said immunomodulatory agent is an immune suppressant. In an even more specific embodiment, said immune suppressive agent is an anti-CD3 antibody (e.g., OKT3, muronomab), an anti-IL-2 receptor antibody (e.g., basiliximab (SIMULECT®) and daclizumab (ZENAPAX®)), an anti T cell receptor antibody (e.g., Muromonab-CD3), azathioprine, a calcineurin inhibitor, a cortico steroid, cyclosporine, methotrexate, mercaptopurine, mycophenolate mofetil, tacrolimus, or sirolimus. In another more specific embodiment, the second therapeutic agent comprises a stem cell of another type, e.g., a bone marrow-derived mesenchymal stem cell, bone marrow, or a hematopoietic stem cell.

In another aspect, the invention provides a method for prevention and/or treatment of ischemic diseases/disorders/conditions, acute tissue injury or degenerative diseases/disorders/conditions, comprising administering the QPSC population to a subject. The cells of the QPSC population of the invention have effective homing ability, including the ability to block the activation of host complements and directed migration to the injury area, both of which offer better cell protection and therapeutic efficacy in vivo.

In another specific embodiment, said ischemic diseases/disorders/conditions is ischemia or ischemic/reperfusion injury of the cardiovascular system, respiratory system, neural system, musculo skeletal system, digestive system, immune system, lymphatic system, endocrine system, exocrine system or integument system.

In another specific embodiment, said acute tissue injury is traumatic, itrogenic, infectious, mechanical or chemical injury of tissues involved in the cardiovascular system, respiratory system, neural system, musculoskeletal system, digestive system, immune system, lymphatic system, endocrine system, exocrine system or integument system.

In another specific embodiment, said degenerative disease/disorder/condition is gene-related, protein-related, single nucleotide polymorphism, cell membrane-associated, or idiopathic functional deterioration of tissues involved in the cardiovascular system, respiratory system, neural system, musculoskeletal system, digestive system, immune system, lymphatic system, endocrine system, exocrine system or integument system.

### EXAMPLES

### Example 1

### Isolation and Cultivation of QPSC Population of the Invention

QPSCs were isolated and purified from subcutaneous adipose tissue of healthy adult donors. Harvested tissues were first centrifuged for 3-10 minutes at 100 RCF to remove the unwanted watery blood, and digested with collagenase for 0.5-6 hours at 37° C. The length of the aforementioned incubation time and appended shaking depend on the tissue condition and size. The isolated QPSCs were then washed with PBS and centrifuged for 3-10 minutes at 400 RCF twice. After dispersion of the pellet, QPSCs were counted and plated on cell culture flasks at a density of lower than 5×10⁶ cells/cm².

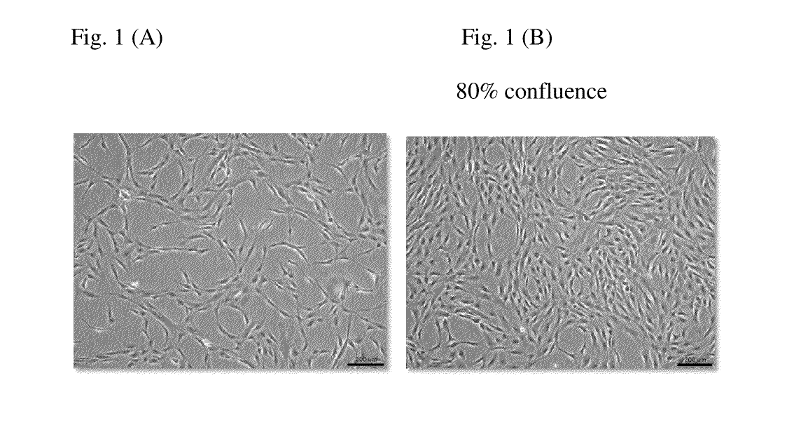

QPSCs were maintained in ADMSC culture medium and the medium was changed every 2-3 days. Once QPSCs reached 70-80% confluence, they were detached with trypsin/EDTA solution for 1-10 minutes at 37° C. followed by washing with PBS and centrifuging for 3-10 minutes at 330 RCF twice. After cell counting, QPSCs were seeded in cell culture flasks at a density of less than 5,000 to 10,000 cells/cm². Seeding density of QPSCs could be adjusted depending upon the length of time interval between subcultures. The cells were cultured in a medium containing 0.1-1.0 ng/ml EGF, 1-10 ng/ml FGF-2 and 0.1-1% ITS. After cultures of six, seven, eight, night, ten, eleven and twelve passages, the QPSC populations were obtained and harvested, respectively. The morphology of QPSC population after 10 passage is shown in FIG. 1.

### Example 2

### Characterization of QPSC Population of the Invention

For characterization of QPSC population of the invention, flow cytometry analysis were performed using a C6 Flow Cytometer® system and the data were analyzed with Accuri CFlow® software. The APC Mouse Anti-Human CD273, FITC Mouse Anti-Human CD46, FITC Mouse Anti-Human CD55, Rabbit Anti-Human CXCR4 & FITC Goat Anti-Rabbit IgG, Human MSC Analysis Kit containing the MSC positive cocktail (FITC CD90, PerCP-Cy™5.5 CD105 and APC CD73), and the QPSC negative cocktail (PE CD45, PE CD34, PE CD11b, PE CD19, and PE HLA-DR) were purchased from BD Bioscience.

For each reaction, 2-5×10⁵ cells of QPSC population were re-suspended in 1% FBS/DPBS solution and incubated with antibodies for 30 minutes on ice. Afterward, QPSC population cells were washed twice with 1 ml 1% FBS/DPBS solution in preparation for flow cytometry analysis.

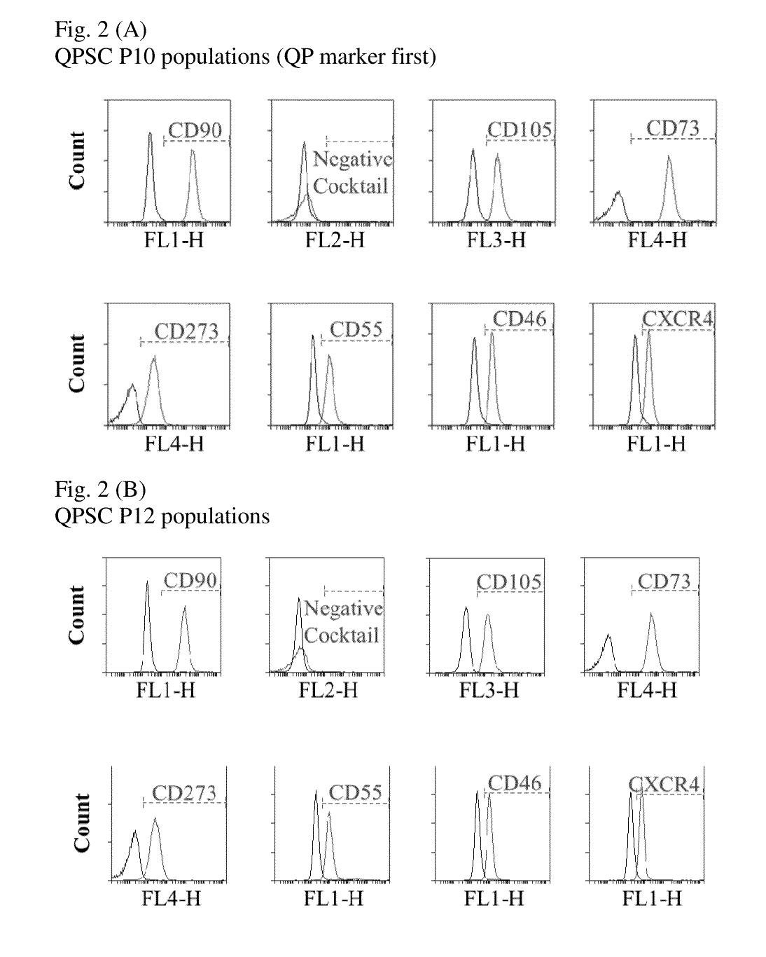

Surface immuno-phenotype characterized by flow cytometry revealed that the QPSC population cells of the invention express CD273, CD55, CD46, CXCR4, CD73, CD90 and CD105 on their surface, while lacking the expression of CD11b, CD19, CD34, CD45 and HLA-DR surface markers (see FIG. 2 and below table).

| | | QPSC population |
|---|---|---|
| | QPSC population | cells of the |
| | cells of the invention | invention (passage |
| Marker | (passage 10; p10) | 12; p12) |
| CD90 | 99.91% | 99.87% |
| CD105 | 99.66% | 99.75% |
| CD73 | 99.94% | 99.85% |
| CD46 | 99.67% | 98.71% |
| CD55 | 98.06% | 98.28% |
| CD273 | 98.59% | 98.75% |
| CXCR4 | 97.90% | 97.97% |
| Negative cocktail | 1.15% | 0.96% |
| (CD11b, CD19, | | |
| CD34, CD45, HLA-DR) | | |

A comparative example is provided to show that the cell marker expression of QPSC population of the invention is different from that of naturally occurring QPSC. The cells were isolated and purified from subcutaneous adipose tissue and cultured to fifth passage according to the methods indicated in Example 1. The expression level of CD273 on the resulting cells was only 60% and the expression level of CD55 and CXCR4 thereof were 40% and 10%, respectively (see the table below).

| | Expression of cell markers on the cells of the |
|---|---|
| Cell marker | comparative example |
| CD273 | 59.73% |
| CD46 | 85.35% |
| CD55 | 34.33% |
| CXCR4 | 5.52% |

### Example 3

### QPSC Population in Trilineage Differentiation Ability

The trilineage differentiation ability of the QPSC population cells of the invention into adipocytes, osteoblasts and chondrocytes was induced with specific cell culture media (STEMPRO® Adipogenesis Differentiation Kit, STEMPRO® Osteogenesis Differentiation Kit, and STEMPRO® Chondrogenesis Differentiation Kit) purchased from Gibco.

For adipogenic differentiation, the cells of QPSC population were seeded on a 35 mm culture dish at a density of 1×10⁵ cells/ml in ADMSC culture medium and incubated at 37° C. in 5% CO₂. After 2-4 hours, the culture medium was replaced with the adipogenesis differentiation medium and the cells of QPSC population were continuously incubated for another 14 or 21 days. Differentiation medium was refreshed every 3-4 days. In order to confirm the adipogenic differentiation of the cells of QPSC population, cells were fixed in 4% formaldehyde solution (Merck) for 20 minutes at room temperature and stained with 40% Oil Red O solution (Sigma) for 5 minutes before observation.

For osteogenic differentiation, the cells of QPSC population were seeded on a 35 mm culture dish at a density of 5×10⁴ cells/ml in ADMSC culture medium and incubated at 37° C. in 5% CO₂. After 2-4 hours, the culture medium was replaced with the osteogenesis differentiation medium and continuously refreshed every 3-4 days. After 14 or 21 days cultivation, cells were fixed in 4% formaldehyde solution for 20 minutes and stained with NBT-BCIP® solution (Sigma) for 45-60 minutes for visualizing alkaline phosphatase activity. Another fresh specimen was stained with Alizarin Red S solution (Sigma) for 60-90 minutes for verifying calcium precipitation.

For chondrogenic differentiation, the cells of QPSC population in ADMSC culture medium of 1×10⁷ cells/ml were collected and 10 μl of the medium were dripped in the center of one well of a 12-well plate following incubation at 37° C. in 5% CO₂ After the cells of OPSC population had adhered for 2 hours on the plate, the chondrogenesis differentiation medium was added and the cells were continuously incubated for another 3 or 6 days. The differentiation medium was carefully refreshed every 2-3 days. In order to verify the chondrogenic differentiation of the cells of QPSC population, cells were fixed in 4% formaldehyde solution for 20 minutes and stained with Alcian Blue solution (Merck) at 4° C. for 2-3 hours before observation.

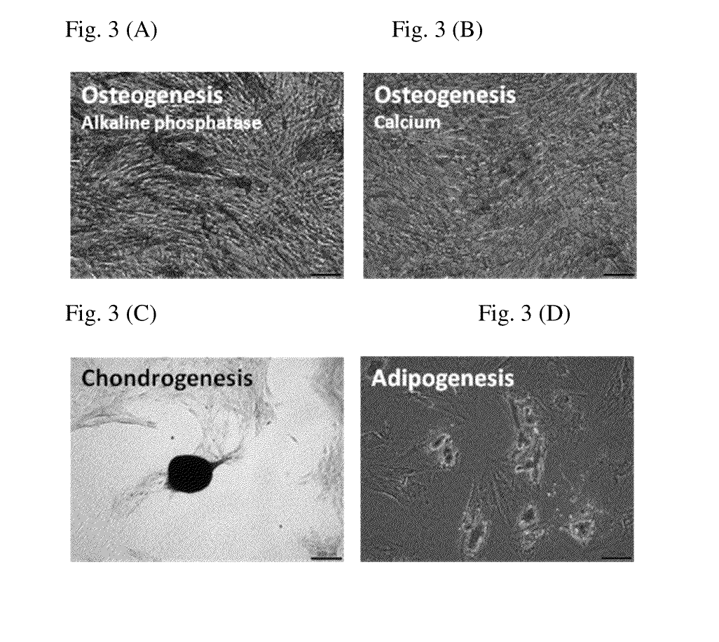

The differentiation ability of the cells of QPSC population of the invention is shown in FIG. 3.

### Example 4

### T Cell Proliferation Assay

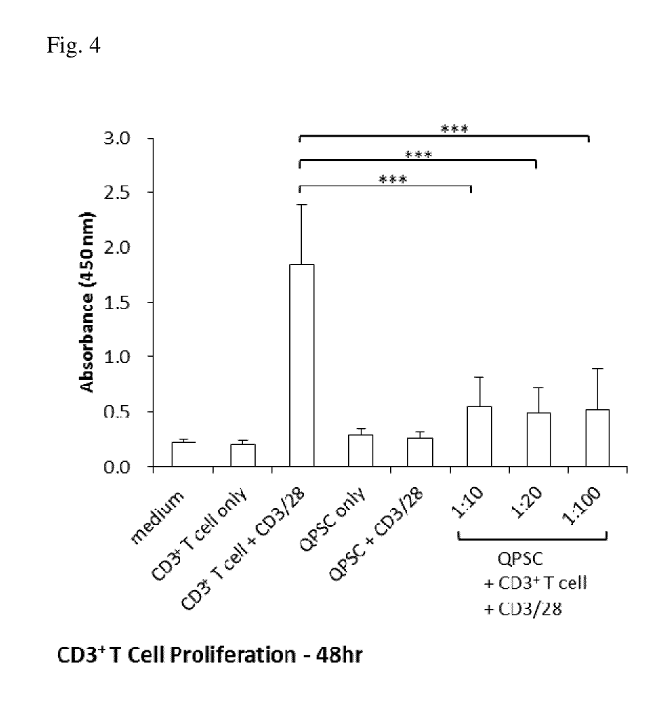

To examine the QPSC immunomodulatory effect on T lymphocyte proliferation, QPSCs were co-cultured at various ratios with freshly isolated T lymphocytes stimulated by plate-bound anti-CD3 antibody plus anti-CD28 antibody. T cell proliferation was then measured using BrdU incorporation method. BrdU (5-bromo-2′-deoxyuridine) is a synthetic thymidine analogue that can be incorporated into newly synthesized DNA in proliferating cells and detected by immunoassay. The results show that T cell proliferation was significantly inhibited by the QPSCs but not in a cell-number-dependent manner (see FIG. 4).

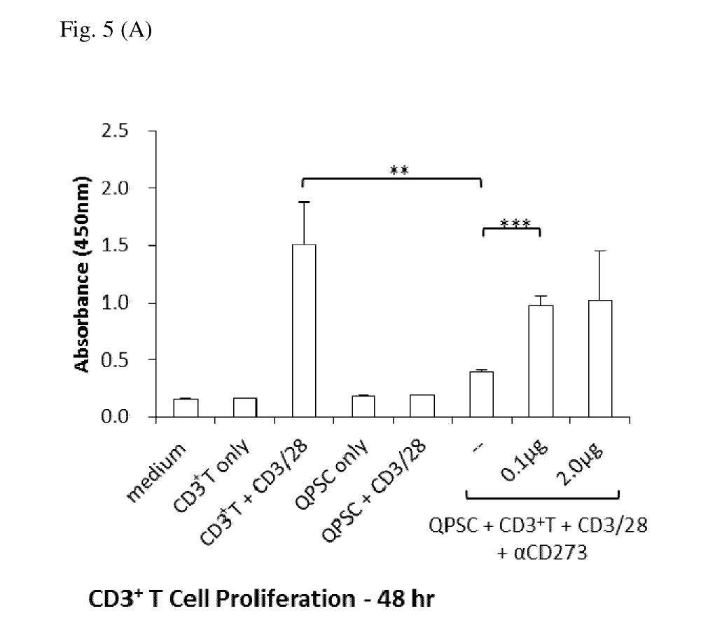

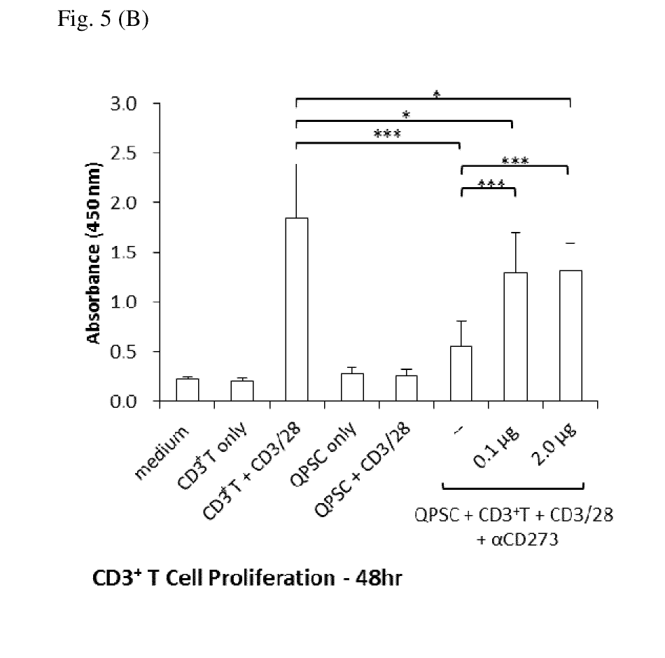

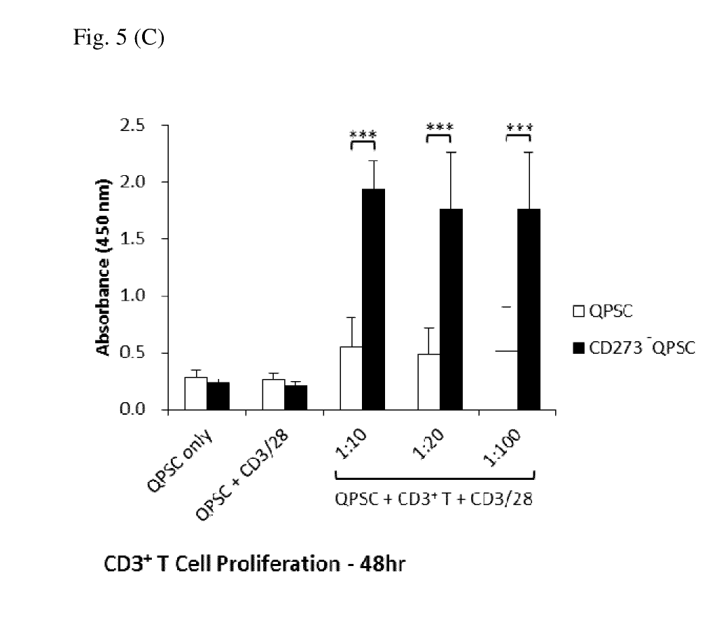

In order to investigate whether CD273 is essential for the immunomodulatory function of QPSC, we compared the T cell modulation ability of QPSCs and CD273-blocked QPSCs (CD273⁻QPSCs) using anti-CD273 neutralizing antibody. QPSC population cells were treated with mytomycin C (50 μg/1×10⁷ cell/ml, Sigma-Aldrich) for 1 hour. CD273-depleted QPSCs were then purified by negative selection using anti-human CD273 antibody conjugated magnetic particles (BD Biosciences) or treated with blocking antibody against CD273 (MIH18, Biolegend) for 1 hour. Effect of CD273 on QPSC population cells for T-cell regulation is shown in FIG. 5. FIG. 5(A) (QPSCs, passage 10) and (B)(QPSCs, passage 12) show that the ability to inhibit T cell proliferation of CD273⁻QPSCs was significantly reduced. In addition, CD273⁻QPSCs were purified by negative selection using anti-human CD273 antibody conjugated magnetic particles. FIG. 5(C) shows that CD273⁻QPSCs failed to suppress T cell proliferation. These results indicate that CD273 expression is important for the immunomodulatory function of QPSCs.

Purified human CD3+ T cells (1×10⁵ cells) were co-cultured with the cells of QPSC population or CD273-depleted cells of QPSC population in RPMI-1640 medium (Gibco) containing 10% fetal bovine serum (FBS), 2 mM 1-glutamine, 100 U/ml penicillin, 100 U/ml streptomycin and 25 mM HEPES. Cells were stimulated with plate-bound anti-CD3 (2 μg/ml) and anti-CD28 (2 μg/ml) monoclonal antibodies (BD Biosciences) in a 96-well plate for 48 and 72 hours (37° C./5% CO₂).

After 2-3 days, 5-bromo-2-deoxyuridine (BrdU) was added to each well and the plate was incubated for another 18 hours for measurement of T cell proliferation. The amount of BrdU incorporated into the T cells was measured using the Cell Proliferation ELISA, BrdU kit (Roche) according to the manufacturer's instructions.

### Example 5

### Cell Marker Expression of QPSC Population Cells of the Invention Before Cryopreservation and after Cryopreservation and Thawing

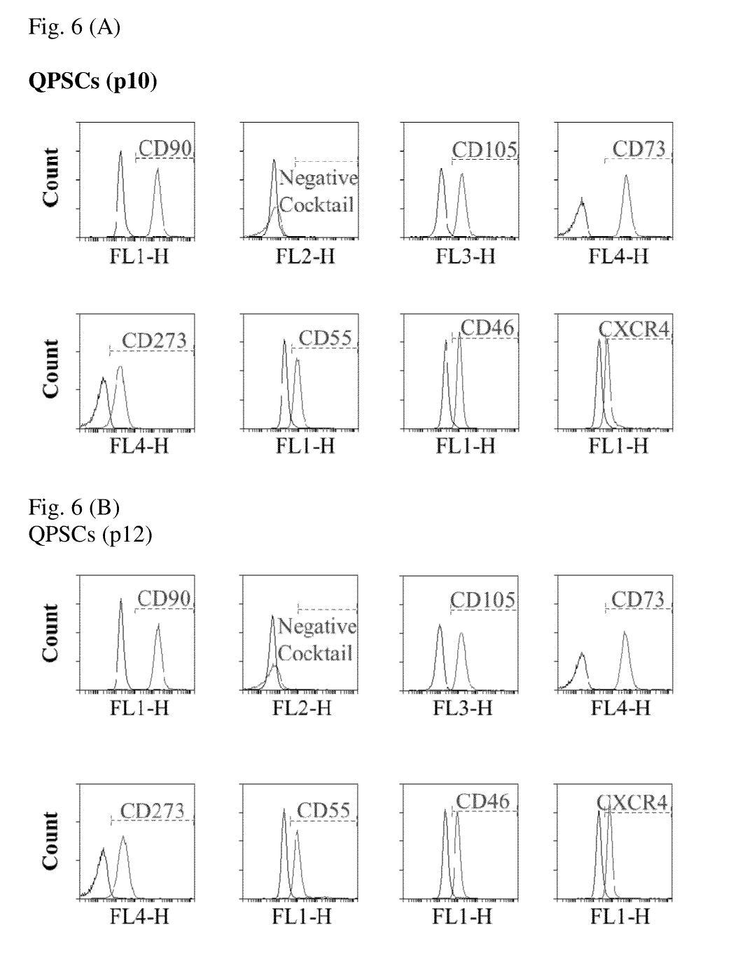

The cell marker expression profile of the cells of QPSC population before cryopreservation is shown in the table below. After using the cryopreservation method described herein, the expression profile of the cell markers of the cells of QPSC population of the invention after cryopreservation and thawing remained substantially identical (see FIG. 6). The expression profiles of the cells of QPSC population before cryopreservation and after cryopreservation and then thawing are shown in the table below.

| | | The cells of | | The cells |
|---|---|---|---|---|
| | | QPSC | | of QPSC |
| | | population | The cells of | population |
| | The cells of | after | QPSC | after |
| | QPSC | cryo- | population | cryo- |
| | population | preservation | before | preservation |
| | before | and then | cryo- | and then |
| Cell | cryopreservation | thawing | preservation | thawing |
| marker | p10 | p10 | p12 | p12 |
| CD90 | 99.91% | 99.89% | 99.87% | 99.88% |
| CD105 | 99.66% | 99.47% | 99.75% | 99.75% |
| CD73 | 99.94% | 99.97% | 99.85% | 99.94% |
| CD46 | 99.67% | 99.24% | 98.71% | 98.67% |
| CD55 | 98.06% | 95.91% | 98.28% | 98.80% |
| CD273 | 98.59% | 97.54% | 98.75% | 98.96% |
| CXCR4 | 97.90% | 82.18% | 97.97% | 98.93% |
| Negative | 1.15% | 0.15% | 0.96% | 0.51% |
| cocktail | | | | |

Negative cocktail: CD11b, CD19, CD34, CD45 and HLA-DR

### Example 6

### In Vitro Chemotaxis Assay

The stromal-derived factor-1 (SDF-1) plays a critical role in cell homing toward ischemic tissue via the interaction with its cognate receptor, CXC chemokine receptor 4 (CXCR4), expressed on the surface of stem/progenitor cells. We examined whether higher expression of CXCR4 increases the mobilization of QPSCs toward an SDF-1 gradient in the transwell chemotaxis assay. For sustained release of chemo-attractant, 100 μl, 2.4 mg/ml hydrogels (Porcogen collagen, SunMax Biotechnology Co., Ltd.) were loaded with 0, 500, and 1000 ng/ml SDF-1α (R&D System) and placed in the bottom well of a 24-well plate and pre-incubated 1 hour at 37° C. for jellification.

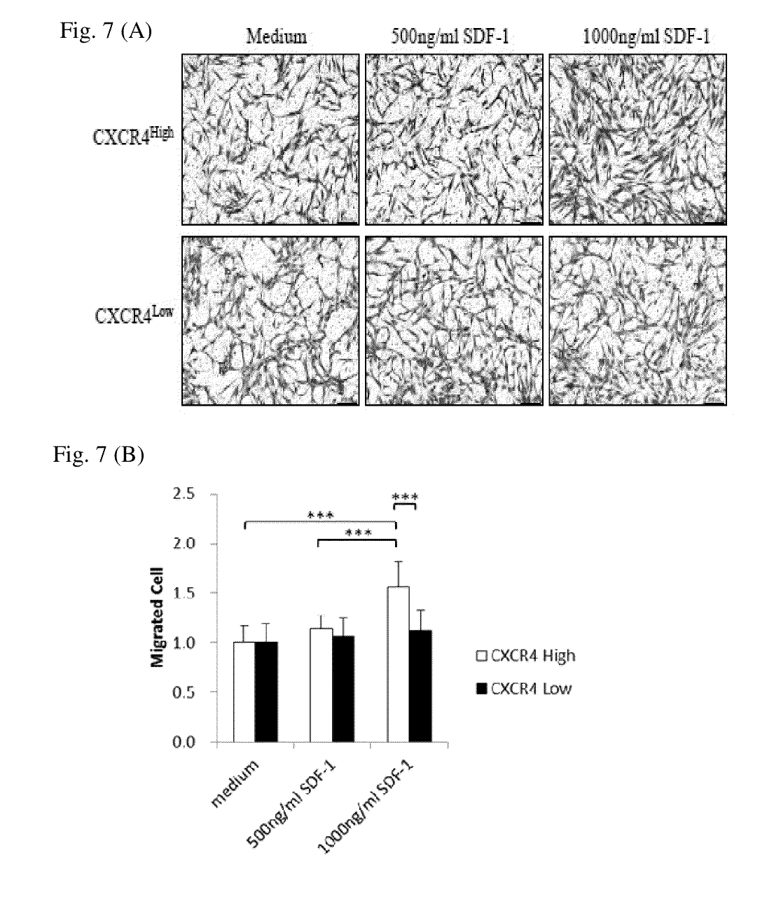

The cells of QPSC population suspended in the Stemchymal Culture Medium (100 μl, 1×10⁵ cells/ml) were loaded onto the upper chamber of a 24-well culture insert (8 μm pore size, PET, BD Falcon™), and an additional 700 μl of the same medium was added into the lower chamber. After overnight pre-incubation at 37° C. and 5% CO₂, the inserts were transferred onto the 24-well plate containing pre-incubated hydrogels, and loaded with 700 μl low serum containing migration medium (0.5% FBS in α-MEM). After 16-24 hours further incubation, cells were fixed with 4% formaldehyde (Merck) for 30 minutes and stained with 0.5% crystal violet solution (Sigma) for 30 minutes. The non-migrating cells on the top side of the insert membrane were removed by gentle swabbing with a cotton tip. The number of migrated cells on the bottom side of the membrane was determined by counting five random fields per well under the microscope at ×40. As shown in FIG. 7, the QPSC population cells have an advantageous homing ability. The results show the cells with higher expression of CXCR4 (CXCR4^{High}) enhances the ability of QPSCs to respond to SDF-1 concentration in a dose-dependent manner. However, the cells with lower expression of CXCR4 (CXCR4^{Low}) do not show increased migrating cell number toward SDF-1.

### Example 7

### Complement-Dependent Cytotoxicity Assay

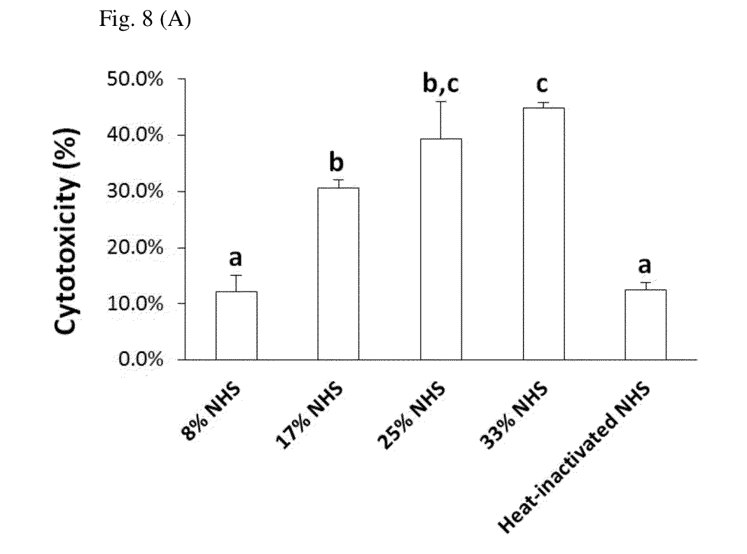

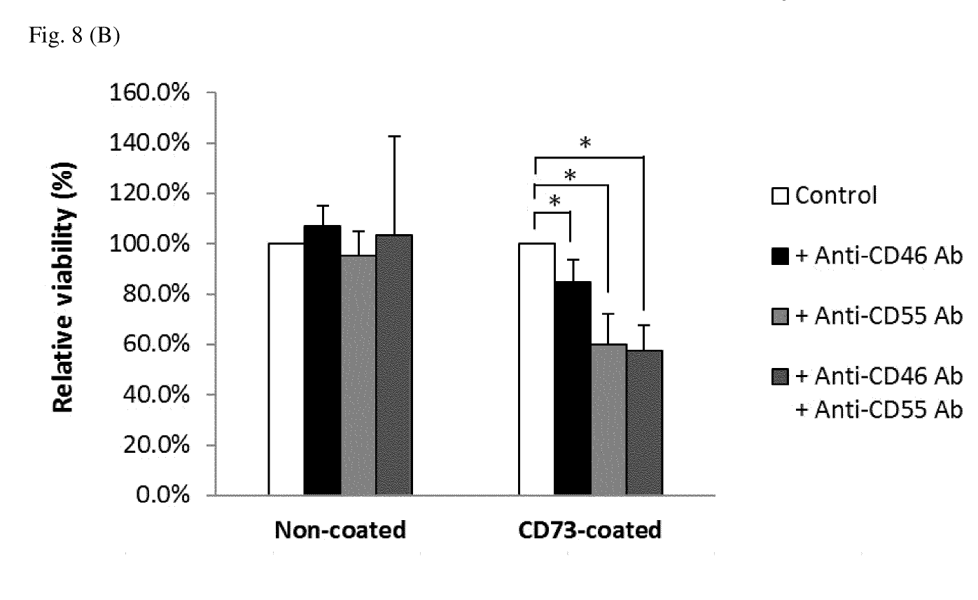

QPSC population cells were seeded in a 96-well tissue culture plate (BD Biosciences) at a concentration of 1×10⁴ cells/well. When attached, cells were incubated with normal human serum collected from healthy adult donors as a source of complement at 37° C. for 1 hr. For positive and negative control, cells were incubated with 1% Triton X-100 (Sigma-Aldrich) and α-MEM (Gibco) respectively. After incubation, MTS assay was used to evaluate cell viability. The MTS assay steps were conducted according to the manufacturer's instructions. Briefly, MTS reagent (Promega) was added into each well of the 96-well tissue culture plate at 1:5 dilution, the plate was incubated at 37° C. for 3 hr in a humidified, 5% CO₂ atmosphere, and the absorbance at 490 nm was recorded by ELISA reader. As shown in FIG. 8, the QPSC population cells have an advantageous ability in regulation of complement-dependent cytotoxicity.

What is claimed is:

1. An isolated and modified QPSC population, which has at least 70% cell homogeneity and expresses at least a cell marker of CD273.

2. The QPSC population of claim 1, which strongly express CD273.

3. The QPSC population of claim 1, which further expresses CD73, CD105 and CD90.

4. The QPSC population of claim 1, which further expresses one or more of CD55, CD46 and CXCR4, while not expressing one or more of CD11b, CD19, CD34, CD45 and HLA-DR.

5. The QPSC population of claim 1, which has at least 90% cell homogeneity and expresses at least a cell marker of CD273.

6. The QPSC population of claim 5, which strongly express CD273.

7. The QPSC population of claim 5, which further expresses CD73, CD105 and CD90.

8. The QPSC population of claim 5, which further expressing one or more of CD55, CD46 and CXCR4, while not expressing one or more of CD11b, CD19, CD34, CD45 and HLA-DR.

9. A composition, comprising the QPSC population of claim 1.

10. A method for producing an isolated and modified QPSC population of claim 1, comprising isolating human multipotent stromal cells from a subject; culturing the resulting cells in a stem cell culture medium; detaching the cells after they reach 70-80% confluence; seeding the cells at a density in the range of 3,000-10,000 cells/cm²; culturing the cells in a medium containing at least one growth factor and a medium supplement; and after cultivation to at least the 4th passage, the cells appear to express CD273 and optionally one or more of CD46, CD55 and CXCR4 and maintain the expression of CD273 and optionally one or more of CD46, CD55 and CXCR4 from at least the 4th passage, whereby an isolated and modified QPSC population can be obtained.

11. The method of claim 10, wherein the seeding density is 5,000 to 10,000 cells/cm².

12. The method of claim 10, wherein the growth factor is EGF, EGF-2 or VEGF or any of their combination and the medium supplement is ITS.

13. The method of claim 12, wherein EGF is in a concentration of 0.1-1.0 ng/ml; FGF-2 is in a concentration of 1-10 ng/ml and ITS is in a concentration of 0.1-1%.

14. A method of maintaining cell markers of QPSCs of claim 1 after large-scale cell cryopreservation and thawing, comprising a step of slowly decreasing the temperature of the large-scale QPSC population cells at a constant cooling rate during the freezing process and then thawing, wherein the temperature difference (ΔT) between the surrounding and center of the large-scale QPSCs ranges from 1.6 to 2.6° C.

15. A method for modulation of T cells and/or complement system and enhancement of cell viability, comprising administering an effective amount of the isolated and modified QPSC population of claim 1 to a subject.

16. A method for enhancement of homing efficiency of cells, comprising administering an effective amount of the isolated and modified QPSC population of claim 1 to a subject.

17. A method for prevention and/or treatment of immune, ischemic, degenerative disease/disorder/condition and acute tissue injury, comprising administering the QPSC population of claim 1 to a subject.

18. The method of claim 17, wherein the immune disease/disorder/condition is inflammatory bowel disease, graft-versus-host disease, rheumatoid arthritis, multiple sclerosis, systemic lupus erythematosus, scleroderma, diabetes or psoriasis.

19. The method of claim 17, wherein the ischemic disease/disorder/condition is ischemia or ischemic/reperfusion injury of the cardiovascular system, respiratory system, neural system, musculoskeletal system, digestive system, immune system, lymphatic system, endocrine system, exocrine system or integument system.

20. The method of claim 17, wherein the acute tissue injury is traumatic, itrogenic, infectious, mechanical or chemical injury of tissues involved in the cardiovascular system, respiratory system, neural system, musculoskeletal system, digestive system, immune system, lymphatic system, endocrine system, exocrine system or integument system.

21. The method of claim 17, wherein the degenerative disease/disorder/condition is gene-related, protein-related, single nucleotide polymorphisms, cell membrane-associated, or idiopathic functional deterioration of tissues involved in the cardiovascular system, respiratory system, neural system, musculo skeletal system, digestive system, immune system, lymphatic system, endocrine system, exocrine system or integument system.

22. The method of claim 17, which comprises further administering a second therapeutic agent.
